SEQUENCE LISTING (9 sequences)

SEQ ID NO: 1 - protein, 116 aa, Homo sapiens
MESSRVRLLPLLGAALLLMLPLLGTRAQEDAELQPRALDIYSAVDDASHEKELIEALQEVLKKLKSKRVPIYEKKYGQVPMCDAGEQCAVRKGARIGKLCDCPRGTSCNSFLLKCL

SEQ ID NO: 2 - protein, 129 aa, Mus musculus
MESSRLRLLPLLGAALLLLLPLLGARAQEDAELQPRALDIYSAVDDASHEKELPRRQLRAPGAMLQIEALQEVLKKLKSKRIPIYEKKYGQVPMCDAGEQCAVRKGARIGKLCDCPRGTSCNSFLLKCL

SEQ ID NO: 3 - protein, 129 aa, Rattus norvegicus
MESSRLRLLPVLGAALLLLLPLLGAGAQEDAELQPRALDIYSAVDDASHEKELPRRQLRAPGAVLQIEALQEVLKKLKSKRIPIYEKKYGQVPMCDAGEQCAVRKGARIGKLCDCPRGTSCNSFLLKCL

SEQ ID NO: 4 - protein, 48 aa, Rattus norvegicus
IPIYEKKYGQVPMCDAGEQCAVRKGARIGKLCDCPRGTSCNSFLLKCL

SEQ ID NO: 5 - protein, 42 aa, Rattus norvegicus
KYGQVPMCDAGEQCAVRKGARIGKLCDCPRGTSCNSFLLKCL

SEQ ID NO: 6 - protein, 41 aa, Rattus norvegicus
YGQVPMCDAGEQCAVRKGARIGKLCDCPRGTSCNSFLLKCL

SEQ ID NO: 7 - protein, 48 aa, Homo sapiens
VPIYEKKYGQVPMCDAGEQCAVRKGARIGKLCDCPRGTSCNSFLLKCL

SEQ ID NO: 8 - protein, 42 aa, Homo sapiens; identical to SEQ ID NO: 5

SEQ ID NO: 9 - protein, 41 aa, Homo sapiens; identical to SEQ ID NO: 6

(19) United States
(12) Patent Application Publication
(10) Pub. No.: US 2010/0075893 A1
(43) Pub. Date: Mar. 25, 2010

(54) PREPARATION OF A PHARMACEUTICAL COMPOSITION FOR INCREASING BONE MINERAL DENSITY

(75) Inventors: Jan Albert Gossen, Oss (NL); Johannes Petrus Leonardus Gerrits, Oss (NL)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon

(21) Appl. No.: 12/407,404

(22) Filed: Mar. 19, 2009

Related U.S. Application Data
(60) Provisional application No. 61/038,296, filed on Mar. 20, 2008.

(30) Foreign Application Priority Data
Mar. 20, 2008 (EP) EP 08005279.8

(51) Int. Cl. A61K 38/00; A61P 19/08

(52) U.S. Cl. 514/2

(57) ABSTRACT

The present invention relates to the use of a cocaine and amphetamine-regulated transcript (CART)-derived peptide for the preparation of a pharmaceutical composition for increasing bone mineral density by continuously providing elevated serum levels of the CART-derived peptide in a subject to be treated. Suitably, the elevated serum levels of the CART-derived peptide are continuously provided through slow release of the CART-derived peptide over an extended period of time from a slow release formulation.

This application is a non-provisional application that claims priority under 35 U.S.C. §119(e) of provisional application U.S. Ser. No. 61/038,296 filed Mar. 20, 2008, the contents of which are hereby incorporated by reference in its entirety.

### BACKGROUND

The present invention relates to the preparation of a pharmaceutical composition for increasing bone mineral density (BMD).

A decrease in bone mineral density occurs for example in osteoporosis. Osteoporosis is an established and well-defined disease that affects more than 75 million people in Europe, Japan and the USA, and causes more than 2.3 million fractures annually in Europe and the USA alone.

Low bone mineral density and micro architectural deterioration of bone tissue lead to an increase in bone fragility. Loss of bone mineral density occurs with advancing age and rates of fracture increase markedly with age, giving rise to significant morbidity and some mortality.

Osteoporosis does not only cause fractures, it also causes people to become bedridden with secondary complications that may be life-threatening in the elderly. Since osteoporosis also causes back pain and loss of height, prevention of the disease and its associated fractures is essential for maintaining health, quality of life, and independence among the elderly.

Osteoporosis is three times more common in women than in men, partly because women have a lower peak bone mass and partly because of the hormonal changes that occur at the menopause. Estrogens have an important function in preserving bone mass during adulthood, and bone loss occurs as levels decline, usually from around the age of 50 years. In addition, women live longer than men and therefore have greater reductions in bone mass.

Osteoporosis itself has no specific symptoms. Its main consequence is the increased risk of fracture. Osteoporotic fractures are those that occur in situations where healthy people would not normally break a bone. Typical fragility fractures occur in the vertebral column, hip and wrist. In addition, the increased risk of falling associated with aging may also lead to fractures.

Various drugs, such as those used in the treatment of breast cancer, are known to cause bone loss. Bone mineral density can also decrease through inactivity, for example caused by bed rest. The bone loss in these cases is often reversible.

It is the object of the present invention to provide a novel therapy for increasing bone mineral density, in particular for use in the treatment of osteoporosis and reversible bone loss, such as resulting from drug use or inactivity.

Cocaine and amphetamine-regulated transcript (CART) is a peptide/neurotransmitter expressed in the central (hypothalamus, pituitary), peripheral and enteric nervous system. In addition, CART is also expressed in the pancreas (endocrine cells), in the gastrointestinal tract (G cells) and in ovary. CART is thought to be involved in feeding, drug reward, stress and cardiovascular function.

CART is furthermore described to play a role in bone resorption in Elefteriou et al., (Nature 434:514-520 (2005)), who identified CART when analyzing mice lacking either leptin ((ob/ob) mice) or its receptor ((db/db) mice). These mutant mice were characterized by phenotypic abnormalities, such as being obese and sterile and having a low sympathetic tone and a high bone mass. CART is mainly expressed in the hypothalamus but expression has also been observed in the pituitary, pancreas and adrenal gland. Expression in hypothalamic neurons is increased by leptin injection and is absent in ob/ob mice. CART knockout mice have no overt phenotypic abnormalities, but are mainly characterized by low bone mass (˜50% reduced BV/TV (bone volume/tissue volume)).

CART expression has also been analyzed in Mc4R^{-/-}mice. Deok Ahn et al. (Endocrinology 147(7):3196-3202 (2006)) disclosed that humans or mice lacking an allele of Mc4r display a decrease in bone resorption parameters, high bone mass and an increase in CART serum levels and/or hypothalamic expression. In addition, it is described that CART signalling accounts for a decrease in bone resorption.

### SUMMARY OF THE INVENTION

In the research that led to the invention it was surprisingly found that treatment of mice with rCART55-102 peptide using a slow release system results in a robust increase of BMD and other relevant bone parameters. Rodents, such as rat and mouse, are a well-accepted animal model for osteoporosis and the results obtained therein may thus be extrapolated to humans.

The above-mentioned literature studies are all directed to the endogenous increase in hypothalamic CART expression of CART. According to the invention it was shown that the peripheral increase of exogenous CART in serum results in an increase in bone mineral density. This could not have been expected in view of the literature described above.

The invention thus relates to the use of a cocaine and amphetamine-regulated transcript (CART)-derived peptide that has the biological activity of human CART for the preparation of a pharmaceutical composition for increasing bone mineral density for the treatment or prevention of bone-mass diseases and for fracture repair, wherein the treatment comprises continuously providing elevated serum levels of the CART-derived peptide in a subject to be treated.

### FIGURES

FIG. 1: Protein alignment of human, mouse and rat CART. * indicates homology between mouse rat and human CART sequence. In bold the human CART42-89 is indicated.

FIG. 2: Trabecular bone mineral density (BMD) data of distal femurs at T=0, 28 and 60 days from control mice and mice implanted with rCART55-102 slow release pellets (*p<0.05; **p<0.01; ***p<0.001).

FIG. 3: Trabecular bone mineral density of distal femur at T=30 days of mice with rCART55-102 slow release pellets or mice treated with daily injections of rCART55-102 peptide.

FIG. 4: Rat CART55-102 induced suppression of FSH-induced 17β-estradiol (E2) production in bovine granulosa cells.

### DETAILED DESCRIPTION

The present invention provides the use of a CART-derived peptide for the preparation of a pharmaceutical composition for increasing bone mineral density and/or bone mass by continuously inducing elevated serum levels of the CART-derived peptide in a subject to be treated.

CART is a neuropeptide that has received much attention as a potential mediator of feeding behaviour and body-weight regulation in mammals. The human CART gene maps to chromosome 5q13-14, a locus that has previously been shown to be a susceptibility locus for obesity.

CART is evolutionarily very well conserved between species (95% amino acid identity between rat and human) with 100% identity in the amino terminal region of the peptide. This region is present in all biologically active CART peptides and contains 6 cysteine residues that are strictly conserved in every species. Disruption of disulfide links results in loss of biological activity.

The full-length prepro-CART of the rat consists of 129 amino acids. Full length human CART comprises 116 amino acids. Without the signal peptide (residues 1-27) the rat pro-CART has either 102 residues (long form) or 89 residues (short) form. To date a number of biologically active CART peptides have been described: CART55-102 and CART62-102 in rodents and CART42-89 and CART49-89 in humans.

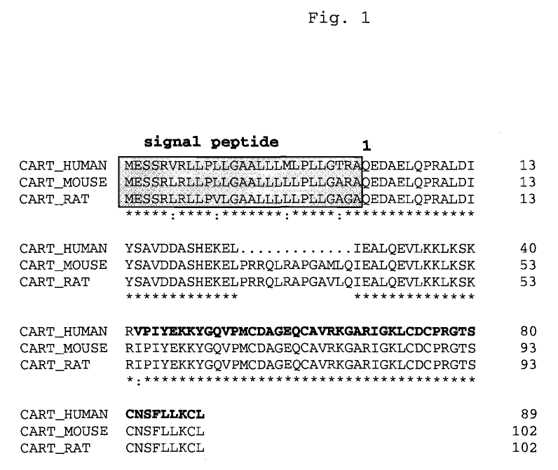

The most widely investigated product of the CART precursor is rCART55-102, which is generally referred to as CART. The biological activity of rCART55-102 has been described in the context of a variety of biological models including e.g. locomotor activity, feeding and anxiety pre-pulse inhibition. Shorter versions have also been shown to retain biological activity but have not been described in detail. In FIG. 1 the protein sequence alignment of human, mouse and rat and the structure of CART are shown.

In an embodiment of the invention, the cocaine and amphetamine-regulated transcript (CART)-derived peptide is the full-length cocaine and amphetamine-regulated transcript (CART) itself, in particular the rat, mouse or human forms of 129 and 116 amino acids, respectively. The full length CART can be obtained either recombinantly (Thim L. et al., FEBS Letters 428:263-268 (1998); Coucero et al., Protein expression and purification 32:185-193 (2003)), or by peptide synthesis (Coucero et al., Protein expression and purification 32:185-193 (2003); Dodson et al., Peptides for the New Millennium, Proceedings of the American Peptide Symposium, 16th, Minneapolis, Minn., United States, Jun. 26-Jul. 1, 1999, Meeting Date 1999, 136-137 (2000)).

In the present application the term CART is used in a broad sense unless otherwise indicated, i.e. it includes full length CART, as well as variants, such as fragments, analogs, derivatives or modifications thereof. Variants are for example CART-derived peptides or proteins wherein one or more amino acid has been substituted by one or more other amino acids or wherein one or more amino acid has been modified or deleted. Suitable amino acid substitutions not likely to change the biological activity of the variant CART are described in for example Dayhof, M. D., Atlas of protein sequence and structure, Nat. Biomed. Res. Found., Washington D.C., 1978, vol. 5, suppl. 3. Amino acid replacements between related amino acids or replacements which have occurred frequently in evolution are, inter alia Ser/Ala, Ser/Gly, Asp/Gly, Arg/Lys, Asp/Asn, Ile/Val. Based on this information Lipman and Pearson developed a method for rapid and sensitive protein comparison (Science 227, 1435-1441, 1985) and determining the functional similarity between homologous polypeptides.

Alternatively, the CART-derived peptide is a fragment, an analog or a derivative of full length human CART or of rat or murine CART that has the biological activity of human CART. In order to determine whether a fragment, analog or derivative still retains the biological activity of human CART the effect of the fragment, analog or derivative on the stimulation of extra-cellular signal-related kinase (ERK) can be tested in the pituitary derived cell lines AtT20 or GH3 (Lakatos et al., Neuroscience Letters 384: 198-202 (2005)), or in bovine granulosa cells; (Sen et al., Molecular Endocrinology 22(12): 2655-2676 (2008)).

In order to still have the biological activity of human CART, treatment with the CART-derived peptide should at least lead to a 1.2-fold increase of P-ERK1/2 at a concentration of 10 μM, preferably a 1.5-fold increase, more preferably a 2-fold increase of P-ERK1/2 activity at a concentration of 0.1 μM in each of the three cell lines as compared to the vehicle without the CART-derived peptide.

Alternatively, the biological activity of the CART-derived peptide can be tested on its ability to suppress FSH-induced 17β-estradiol (E2) production in bovine granulosa cells as described in Example 3 and by Sen et al. (Endocrinology 148(9):4400-4410 (2007)). Treatment with the CART-derived peptide should at least result in a 1.2-fold decrease, preferably a 1.5-fold decrease, more preferably a 2-fold decrease, and even more preferably a 3-fold decrease of FSH-induced 17β-estradiol production at a concentration of the CART-derived peptide of 1 μM. Most preferably, treatment with the CART-derived peptide leads to a 1.5-fold decrease in E2 production at a concentration of 1 nM as compared to vehicle.

Final proof that the CART activity according to the invention is maintained can be obtained by repeating the experiment as described in Example 1.

Rat CART fragments desirably incorporate at least the amino acids 55-102, 61-102 or 62-102. Human CART fragments desirably incorporate at least the amino acids 42-89, 48-89 and 49-89. The amino acid sequences of such CART fragments are as follows:

Rat CART fragment 55-102
Ile-Pro-Ile-Tyr-Glu-Lys-Lys-Tyr-Gly-Gln-Val-Pro-
Met-Cys-Asp-Ala-Gly-Glu-Gln-Cys-Ala-Val-Arg-Lys-
Gly-Ala-Arg-Ile-Gly-Lys-Leu-Cys-Asp-Cys-Pro-Arg-
Gly-Thr-Ser-Cys-Asn-Ser-Phe-Leu-Leu-Lys-Cys-Leu
Rat CART fragment 61-102
Lys-Tyr-Gly-Gln-Val-Pro-Met-Cys-Asp-Ala-Gly-Glu-
Gln-Cys-Ala-Val-Arg-Lys-Gly-Ala-Arg-Ile-Gly-Lys-
Leu-Cys-Asp-Cys-Pro-Arg-Gly-Thr-Ser-Cys-Asn-Ser-
Phe-Leu-Leu-Lys-Cys Leu
Rat CART fragment 62-102
Tyr-Gly-Gln-Val-Pro-Met-Cys-Asp-Ala-Gly-Glu-Gln-
Cys-Ala-Val-Arg-Lys-Gly-Ala-Arg-Ile-Gly-Lys-Leu-
Cys-Asp-Cys-Pro-Arg-Gly-Thr-Ser-Cys-Asn-Ser-Phe-
Leu-Leu-Lys-Cys-Leu
Human CART fragment 42-89
Val-Pro-Ile-Tyr-Glu-Lys-Lys-Tyr-Gly-Gln-Val-Pro-
Met-Cys-Asp-Ala-Gly-Glu-Gln-Cys-Ala-Val-Arg-Lys-
Gly-Ala-Arg-Ile-Gly-Lys-Leu-Cys-Asp-Cys-Pro-Arg-
Gly-Thr-Ser-Cys-Asn-Ser-Phe-Leu-Leu-Lys-Cys-Leu
Human CART fragment 48-89
Lys-Tyr-Gly-Gln-Val-Pro-Met-Cys-Asp-Ala-Gly-Glu-
Gln-Cys-Ala-Val-Arg-Lys-Gly-Ala-Arg-Ile-Gly-Lys-
Leu-Cys-Asp-Cys-Pro-Arg-Gly-Thr-Ser-Cys-Asn-Ser-
Phe-Leu-Leu-Lys-Cys-Leu
Human CART fragment 49-89
Tyr-Gly-Gln-Val-Pro-Met-Cys-Asp-Ala-Gly-Glu-Gln-
Cys-Ala-Val-Arg-Lys-Gly-Ala-Arg-Ile-Gly-Lys-Leu-
Cys-Asp-Cys-Pro-Arg-Gly-Thr-Ser-Cys-Asn-Ser-Phe-
Leu-Leu-Lys-Cys-Leu

Also part of the invention is the use of CART-derived peptides that show at least 90%, preferably at least 95%, more preferably at least 98% and most preferably at least 99% identity with the above listed peptide sequences. The percentage of sequence identity as used herein is the number of identical amino acids between the CART-derived peptide and a reference sequence. Suitable reference sequences are the RefSeq peptides: NP_{—}004282.1 (human), NP_{—}001074962.1 (mouse isoform 2), NP_{—}038760.3 (mouse isoform 1), NP_{—}058806.1 (rat). These sequences can be found in the RefSeq database, a public database of nucleotide and protein sequences with corresponding feature and bibliographic annotation from the National Center for Biotechnology Information (NCBI).

Alternative CART-derived peptides in the form of CART variants have substituted, modified and/or deleted amino acids. Modifications are preferably such that the disulfide bonds between cysteine residues 68-86, 74-94 and 88-101 in rat CART and between cysteine residues 55-73, 61-81 and 75-88 in human CART are maintained intact and do not interfere with biological activity of CART (see FIG. 1).

Increasing the bone mineral density according to the invention is preferably for the treatment or prevention of osteoporosis (including postmenopausal osteoporosis in women and idiopathic and primary hypogonodal osteoporosis in men), glucocorticoid-induced osteoporosis, drug-induced bone loss (including estrogen deficiency induced bone loss as a result of the use of e.g. aromatase inhibitors and GnRH receptor modulators or as a result of anorexia nervosa), disuse- or immobilization-induced bone loss, periodontitis-induced bone loss, bone fractures (i.e. bone fracture healing), back pain in postmenopausal women, osteogenesis imperfecta.

The treatment according to the invention is suitably effected by continuously providing elevated serum levels of the CART-derived peptide in a subject to be treated, in particular through slow release thereof over an extended period of time.

Slow release of the CART-derived peptide can be achieved by including the peptide in a slow release formulation. Such slow release formulations for use in humans are known in the art and are for example based on biodegradable polymeric matrices (Chan Y P et al., Expert Opin Drug Deliv. 4(4):441-51 (2007); Shimizu T et al., Biochem Biophys Res Commun. 367(2):330-5 (2008)).

An alternative formulation is for example an implant or a vaginal ring. Implants and vaginal rings are known to the person skilled in the art and are for example described in Power J et al., Cochrane Database Syst Rev. 18(3):CD001326 (2007); De Leede L G et al., Contraception 34(6):589-602 (1986); and Reddy K V et al., Reproduction 128:117-126 (2004).

As used herein “extended period of time” comprises 1-3 months, preferably 3-6 months, more preferably 6-12 months, even more preferably 12-24 months.

For achieving an increase in bone mineral density, the serum levels of the CART-derived peptide are increased at least 1.3-fold, preferably at least 2-fold, more preferably at least 3-fold and up to 5-fold as compared to the endogenous serum levels of CART in the subject to be treated. In one embodiment the serum levels of CART are between 30 and 650 pg/ml.

It was found according to the invention that the therapeutic effect on the bone mineral density is obtained when the serum levels of CART are continuously elevated. This can be achieved by continuous release of the CART-derived peptide in serum or by avoiding or inhibiting clearance of the CART-derived peptide.

As used herein an “increase in bone mineral density (BMD)” comprises a 1.05-fold, preferably a 1.3-fold, more preferably a 1.5-fold, even more preferably a 2-fold increase of the BMD value as determined with a DEXA-scan as compared to placebo treated subjects. Dual energy X-ray absorptiometry (DEXA) is a means of measuring bone mineral density (BMD) and is the most widely used and most thoroughly studied bone density measurement technology.

The CART-derived peptide for use according to the invention can typically be administered in the form of slow release formulations, in particular slow release implants. In this form only one dose is required every month, every few months or every 1 or 2 years.

The invention further relates to a pharmaceutical composition comprising a cocaine and amphetamine-regulated transcript (CART)-derived peptide and a suitable excipient. The pharmaceutical composition is preferably a slow release formulation. For making slow release formulations, the use of a slow release matrix, such as a biodegradable polymeric matrix and optionally conventional additives is contemplated. In general, any pharmaceutically acceptable additive which does not interfere with the function of the CART-derived peptide can be used in the subject invention.

The invention according to a further aspect thereof provides an implant that contains the CART-derived peptide. In another embodiment, the invention relates to a vaginal ring that contains the CART-derived peptide. The implant and vaginal ring suitably comprise the CART-derived peptide contained in a slow release matrix.

The invention furthermore relates to a method for increasing bone mineral density, comprising continuously providing elevated serum levels of the CART-derived peptide in a subject to be treated. The invention also relates to a method for the treatment or prevention of osteoporosis (including postmenopausal osteoporosis in women and idiopathic and primary hypogonodal osteoporosis in men), glucocorticoid-induced osteoporosis, drug-induced bone loss (including estrogen deficiency induced bone loss as a result of the use of e.g. aromatase inhibitors and GnRH receptor modulators or as a result of anorexia nervosa), disuse- or immobilization-induced bone loss, periodontitis-induced bone loss, bone fractures (i.e. bone fracture healing), back pain in postmenopausal women, osteogenesis imperfecta.

The present invention will be further illustrated in the Examples that follow and which are in no way intended to limit the invention.

### EXAMPLES

### Example 1

### Bone Mineral Density Increase in Mice

Bone Mineral Density Increase in Mice by Slow Release CART Treatment

In this experiment mice were treated with 3 different concentrations of CART peptide. The experiment was conducted in 12 weeks old C57B1/6J females and consisted of four experimental groups (n=5 per group). The first group of mice received placebo slow release pellets (SX-999, 60 day slow release pellets, Innovative Research of America, Sarasota, Fla.). The other three groups were treated with SX-999 slow release pellets containing either 20 μg, 50 μg or 100 μg (2×50 μg) rat CART 55-102 peptide (rCART) (Phoenix Pharmaceuticals, Inc. Belmont, Calif.). Mice were sedated with injection anaesthetics (Hypnorm (VetaPharma, Leeds, UK)/Dormicum (Roche, Mijdrecht, The Netherlands)) and the pellets were implanted subcutaneously in the neck.

Bone architecture and density was determined at the distal femur at T=0 by an in vivo micro-CT measurement (Skyscan1076 micro-CT scanner, Skyscan, Kontich, Belgium). Scans were performed at 9 μm resolution. Trabecular bone analysis was performed on a 2 mm slice 150 μm proximal to the distal growth plate of the femur. In vivo micro-CT measurements were repeated at day 28 and day 60. The BMD value of the placebo group at T=0, T=28 and T=60 days was set at 100% and the BMD values of the 20 μg, 50 μg and 100 μg (2×50 μg) CART treated groups were calculated relatively to the placebo group. Values are expressed as mean±SD of the individual data. For statistical analysis the treatment groups were compared with the placebo group of the corresponding time point in a standard Student T-test (two-tailed). P<0.05 was considered as significant.

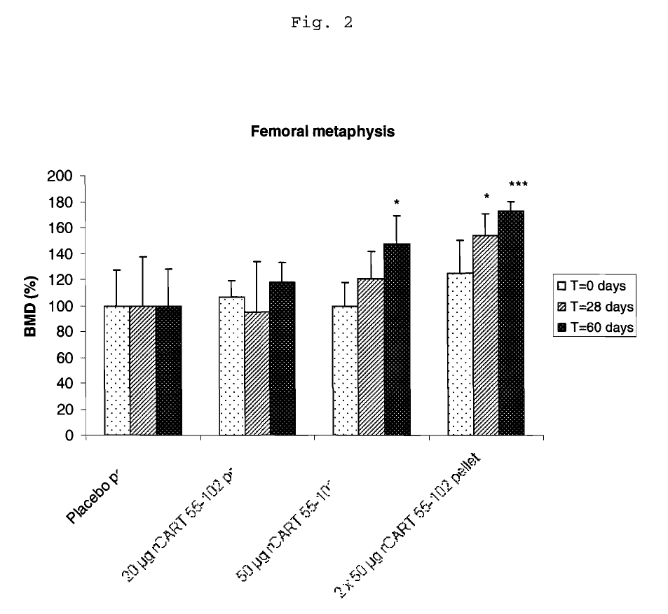

The results are shown in FIG. 2. The results showed a 50-60% increase in trabecular bone mineral density (BMD) after 28 days in the 100 μg CART group as compared to the placebo group. After 60 days BMD has increased further in the 100 μg CART group and was now also significantly increased in the 50 μg CART group. BMD was increased by 80% and 40-50% in the 100 μg and 50 μg groups, respectively.

These data show that continuous release of CART peptide for 28 days, through slow release implant delivery, is sufficient to increase trabecular BMD by 50%. This demonstrates the potential of peripherally administered CART peptide in the treatment or prevention of osteoporosis.

### Example 2

### CART Slow Release Formulation vs. Daily CART Injections

In this experiment the effectiveness of CART peptide to improve bone mineral density via a slow release formulation was compared with daily injections of CART. The experiment was conducted in 12 weeks old C57B1/6J females and consisted of four experimental groups (n=5 per group). In the first group of mice 2 placebo slow release pellets (SX-999, 60 day pellets, Innovative Research of America, Sarasota, Fla.) were implanted subcutaneously in the neck. In the second group 2 SX-999 slow release pellets containing 50 μg rat CART55-102 (Phoenix Pharmaceuticals, Inc. Belmont, Calif.) each, were implanted subcutaneously in the neck. The other two groups received daily s.c. injections with either vehicle (0.9% NaCl) or with 2 μg rat CART55-102 peptide in 0.9% NaCl.

Mice were sedated with injection anaesthetics (Hypnorm (VetaPharma, Leeds, UK)/Dormicum (Roche, Mijdrecht, The Netherlands)) and bone density was determined in the distal femur by an in vivo micro-CT measurement (Skyscan1076 micro CT scanner, Skyscan, Belgium) at T=0 and at T=30 days. Blood was collected weekly by orbita puncture to determine serum CART levels. BMD (g/cm³) was expressed as mean±SD of the individual data. Statistical analysis was performed using the Student T-test (two-tailed). P<0.05 was considered as significant.

The results are shown in FIG. 3. The results show that the mice treated with CART slow release pellets displayed a 50% increase in trabecular bone mineral density (BMD) after 30 days as compared to the placebo pellet group. The group that received daily injections with 2 μg of rat CART 55-102 did not show an increase in BMD. These data show that continuous increase in CART serum levels for 30 days, through slow release implant delivery, does significantly increase trabecular BMD.

### Example 3

### CART Induced Suppression of FSH-Induced 17β-Estradiol Production in Bovine Granulosa Cells

Ovaries from double muscled cows were obtained at a local abattoir and granulosa cells were collected from 3- to 5-mm follicles at random stages of the estrus cycle. Follicles were placed in culture medium (M505 medium (Gibco Invitrogen, Carlsbad, Calif., USA) supplemented with antibiotics (100 IU/ml penicillin and 0.1 mg/ml streptomycin (Invitrogen, Carlsbad, Calif., USA), 10 ng/mL insulin (Schering-Plough, Oss, The Netherlands), 4 ng/mL sodium selenite (Sigma Aldrich, St. Louis, Mo., USA), 5 μg/mL apo-transferrin (Sigma Aldrich, St. Louis, Mo., USA) and 10⁻⁷M androstenedione (Org 6, Schering-Plough, Oss, The Netherlands)) at 37° C. Follicles were ruptured with a needle and the cells were passed through a cell strainer (70 μm nylon, Becton Dickinson, Bedford, USA). Cells were centrifuged for 10 min at 340×g and resuspended in culture medium. Cells (1×10⁵ viable cells/well) were cultured in 96 wells plate for 7 days at 37° C. in a humidified atmosphere (5% CO₂, 95% air) in the presence of 0, 20, 100 or 300 mUnits FSH (Org 32489, Schering-Plough, Oss, The Netherlands) in combination with 0, 10⁻⁸, 10⁻⁷, 10⁻⁸, 10⁻⁹, 10⁻¹⁰, 10⁻¹¹ or 10⁻¹²M rat CART 55-102 (Phoenix Pharmaceuticals Inc., Belmont, Calif., USA). Culture medium was refreshed every 48 h. After 7 days 17β-estradiol levels were determined in the culture medium using a 17β-estradiol ELISA (Diagnostic Systems Laboratories Inc., Marburg, Germany).

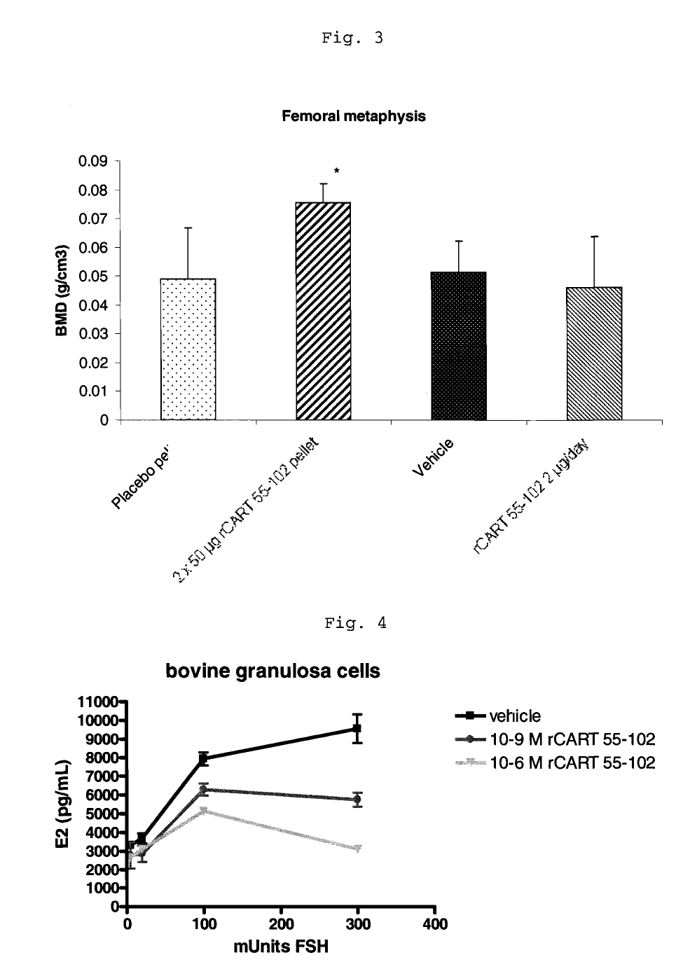

The results are presented in FIG. 4 and show that 1 μM CART peptide can suppress FSH-induced 17β-estradiol production of bovine granulosa cells 1.5-fold in the presence of 100 mUnits FSH and 3-fold in the presence of 300 mUnits of FSH.

1. A pharmaceutical composition for increasing bone mineral density comprising a cocaine and amphetamine-regulated transcript (CART)-derived peptide that has the biological activity of human CART and a suitable excipient in a slow release formulation.

2. The pharmaceutical composition according to claim 1, wherein the CART-derived peptide comprises one or more of human CART42-89, human CART48-89 and human CART49-89.

3. A method for increasing bone mineral density in a subject in need thereof, comprising continuously providing elevated serum levels of a cocaine and amphetamine-regulated transcript (CART)-derived peptide that has the biological activity of human CART to the subject.

4. The method according to claim 3, wherein bone mineral density is increased for the treatment or prevention of bone-mass diseases and for fracture repair.

5. The method according to claim 3, wherein the elevated serum levels of the CART-derived peptide are continuously provided through slow release of the CART-derived peptide over an extended period of time from a slow release formulation.

6. The method according to claim 5, wherein the slow release formulation is in the form of an implant.

7. The method according to claim 6, wherein the implant is a subcutaneous implant.

8. The method according to claim 5, wherein the slow release formulation is in the form of a vaginal ring.

9. The method according to claim 5, wherein the slow release formulation is in the form of a biodegradable polymeric matrix.

10. The method according to claim 3, wherein the elevated serum levels of the CART-derived peptide are continuously provided by avoiding or inhibiting clearance of the CART-derived peptide.

11. The method according to claim 3, wherein the CART-derived peptide is selected from the group consisting of full-length CART, rat CART55-102, rat CART61-102, rat CART62-102, human CART42-89, human CART48-89 and human CART49-89.

12. The method according to claim 3, wherein increasing the bone mineral density is for the treatment or prevention of a disease/condition selected from the group consisting of osteoporosis, drug-induced bone loss, disuse- or immobilization-induced bone loss, periodontitis-induced bone loss, bone fractures, back pain in postmenopausal women and osteogenesis imperfecta.

13. The method according to claim 12, wherein the disease/condition is osteoporosis.

14. The method according to claim 13, wherein the osteoporosis is selected from the group consisting of postmenopausal osteoporosis, idiopathic and primary hypogonodal osteoporosis and glucocorticoid-induced osteoporosis.

15. The method according to claim 5, wherein the extended period of time is 1-3 months.

16. The method according to claim 5, wherein the extended period of time is 3-6 months.

17. The method according to claim 5, wherein the extended period of time is 6-12 months.

18. The method according to claim 5, wherein the extended period of time is 12-24 months.

19. The method according to claim 3, wherein the elevated serum levels of the CART-derived peptide are increased at least 1.3-fold as compared to the endogenous serum level of CART.

20. The method according to claim 3, wherein the elevated serum levels of the CART-derived peptide are increased at least 3-fold as compared to the endogenous serum level of CART.

21. The method according to claim 3, wherein the elevated serum levels of the CART-derived peptide are increased at least 5-fold as compared to the endogenous serum level of CART.

22. The method according to claim 3, wherein the elevated serum levels of the CART-derived peptide are between 30 and 650 pg/ml serum.

23. The method according to claim 3, wherein the increase in bone mineral density is determined by Dual energy X-ray absorptiometry (DEXA) as compared to placebo treated subjects.
